(19) United States
(12) Patent Application Publication
(10) Pub. No.: US 2007/0264370 A1
(43) Pub. Date: Nov. 15, 2007

(54) Composition and methods for the treatment of joint pain using Angelica gigas Nakai extract and powder as combined with Glucosamine Sulfate, or Chondroitin Sulfate and HCL, or MSM, or aspirin, or Celedrin, and as combinations thereof in powder, pill, capsule, spray, liquid, and gelcap form

(76) Inventor: Michael Gregory Jeffers, Bloomington, IN (US)

Correspondence Address: MICHAEL G. JEFFERS, 5721 KAM LANE, BLOOMINGTON, IN, 47408, US

(21) Appl. No.: 11/731,434

(22) Filed: Mar. 30, 2007

Related U.S. Application Data
(60) Provisional application No. 60/788,015, filed on Mar. 31, 2006.

(51) Int. Cl. A61K 36/232; A61P 19/02

(52) U.S. Cl. 424/773

(57) ABSTRACT

The present invention relates to composition comprising dry powder of decursinol, decursin, and their related derivates isolated from the roots of Angelica gigas Nakai (Korean Angelica) plant and at least one of joint improvement agents such as glucosamine, chondroitin, or methylsulfonylmethane (MSM). The present invention is directed to methods, combinations, and compositions for treating, preventing or reducing the joint pain or the symptoms associated with, or related to, a joint disorder or disease in a subject in need thereof.

### FIELD OF THE INVENTION

The present invention is related to compositions comprising an extract of plant Angelica gigas Nakai (Korean angelica) and one or more joint improvement agent(s) comprising Glucosamine, Chondroitin, and MSM. In addition, methods for manufacture of the formulations and uses of the formulations in treating joint pains are described.

### BACKGROUND OF THE INVENTION

Nearly 60 million US adults are suffering from a form of joint disorders. Although there are literally hundreds of different kinds of arthritis, osteoarthritis, rheumatoid arthritis and gout are the three most common. These joint disorder and related diseases can manifest in symptoms including soft tissue erosion, inflammation, swelling, redness, pain and stiffness. As far as treatments are concerned, until recently the most popular have been acetaminophen and the NSAIDs (Non-Steroidal Anti-Inflammatory Drugs) such as ibuprofen, naproxen, aspirin, coxibs (Vioxx®, Celebrex® and Bextra®).

Conventional analgesics such as acetaminophen and non-steroidal anti-inflammatory drugs (NSAID) such as ibuprofen, naproxen, aspirin, and coxibs (selective COX-2 inhibitors) are associated with side effects including mucosal damage, hepatotoxicity, and other undesirable side effects.

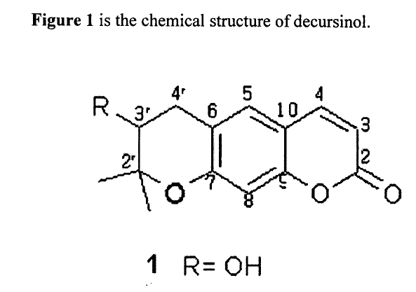

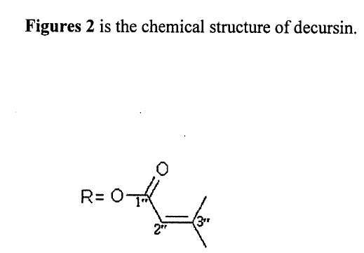

Decursinol (FIG. 1) and decursin (FIG. 2) are two main ingredients of the extract of the roots of Angelica gigas or Korean Angelica (Chi H-J et al., Kor. J. Pharmacology, 1: 25-32 (1970). Kim et al. teaches the use of decursinol and its derivative thereof, isolated from the plant as natural analgesics. See U.S. patent application Ser. No. 09/936,947 (abandoned in 2004).

Decursinol or extract of Korean Angelica is also known for preventing dementia in aged population. See U.S. Patent Application No. 1999/13613. Furthermore, decursin isolated from Angelica gigas Nakai (Korean angelica) is reported to act as an anti-cancer agent (Yim et al., A Novel Anticancer Agent, Decursin Induces G1 Arrest and Apoptosis in Human Prostate Carcinoma Cells, Cancer Research, 65:1035-1044 (2005),).

Glucosamine, also called chitosamine, is an aminomonosaccharide, a form of amino sugar, a component of almost all human tissues, including cartilage. Glucosamine is a natural substance that is found in the shells of shellfish or refined from corn. It is available in different forms, including glucosamine hydrochloride, N-acetyl-glucosamine (NAG), and glucosamine sulfate, which is a combination of glucosamine and mineral salt. Glucosamine sulfate is used as a joint improvement agent. e.g., See Towheed T E, Anastassiades T P. Glucosamine therapy for osteoarthritis. Editorial. J Rheumatol, 26:2294-2297 (1999).

Glucosamine is a natural product that has been used for the alleviation of the symptoms of arthritis and has been shown to be extremely beneficial for relieving joint pain. It appears that glucosamine may be involved in the production of cartilage, which is a key component for healthy joints. Glucosamine is the principle component of glycosaminoglycans, the material that forms the matrix of all connective tissues. Glycosaminoglycans, together with proteoglycans, the mesh-like tissue that permits cartilage to flex and absorb physical shock, yield healthy joints. Glucosamine stimulates cartilage cells to synthesize glucosaminoglycans and proteoglycans, which are responsible for maintaining the human body's joints. Glucosamine appears to also inhibit the production of the enzymes that break down and destroy cartilage, known as chondrocytes (Setnikar, et al. Antereactive properties of glucosamine sulfate., Arzneimittel Forschung, 41:157-161, 1991). Glucosamine has been shown to relieve joint pain, rejuvenate the synovial fluid (fluid within the joint), and promote healthy and flexible joints. See, e.g., Houpt J B, McMillan R, Wein C, Paget-Dello S D. Effect of glucosamine hydrochloride in the treatment of pain of osteoarthritis of the knee. J Rheumatol. 1999; 26:2423-2430.

Chondroitin, also known as chondroitin sulfate, chondroitin sulfuric acid, or chonsurid, occurs naturally in human cartilage, bone, cornea, skin and the arterial wall. In addition, Chondroitin can come from natural sources, such as shark or bovine cartilage, or can be made in a lab. Chondroitin is part of a large protein molecule (proteoglycan) that gives cartilage elasticity. Chondroitin has been used for the alleviation of the symptoms of arthritis . It has been reported that chondroitin appears to be involved in the production of cartilage that is important for healthy joints. See, e.g., McAlindon et al., Glucosamine and chondroitin for treatment of osteoarthritis. A systematic quality assessment and meta-analysis. JAMA. 2000; 283:1469-1475.

Methylsulfonylmethane (MSM) is a naturally occurring, organic sulfur-containing compound related to DMSO (Dimethyl Sulfoxide). MSM (methylsulfonylmethane) is a non-metallic organic compound that plays an essential role in human nutrition. When amino acids, the trace minerals zinc, copper and silicon and vitamin C are present, the body metabolizes MSM to sulfur (Kocsis J J, Harkaway S, Snyder R. Biological effects of the metabolites of dimethyl sulfoxide. Ann NY Acad Sci. 1975; 243:104-109). Sulfur, a structural component integral to new cell growth, is stored in every cell of the body, particularly the hair, nails, bones, teeth and the connective tissue of joints and skin, where it is an important component of protein. Herschler disclosed the use of MSM to enhance the health of an animal including human beings. See U.S. Pat. No. 5,071,878, Use of methylsulfonylmethane to enhance diet of an animal. Other studies have reported that joints affected by osteoarthritis have lower sulfur content and mice with arthritis given MSM experience less joint deterioration (Layman et. al., Growth inhibitory effects of dimethyl sulfoxide and dimethyl sulfone on vascular smooth muscle and endothelial cells in vitro. In Vitro Cell Dev Biol. 23:422-428, (1987)).

When Decursinol and decursin from the extract of the roots of Angelica gigas (Korean Angelica) are co-administered with one or more of joint improvement agents such as glucosamine, chondroitin, or MSM, it has synergetic property with these agents by providing immediate pain relief while assisting the action of improving the joint repair action of these agents.

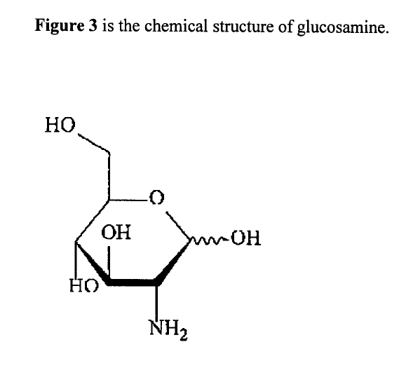

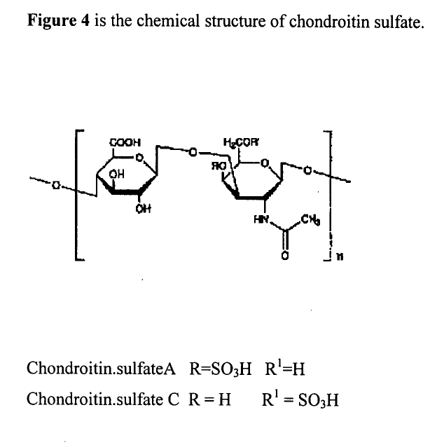

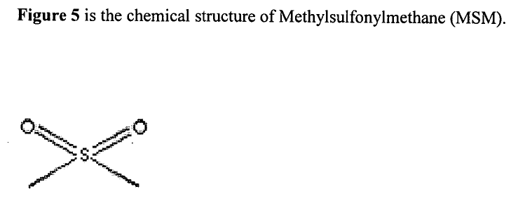

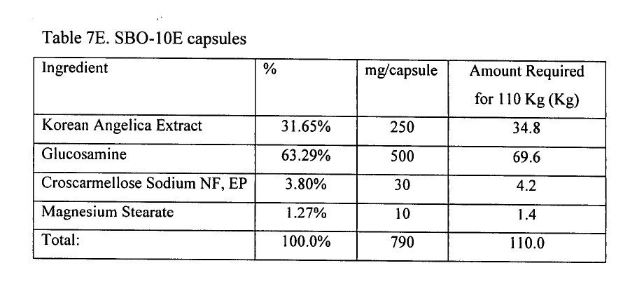

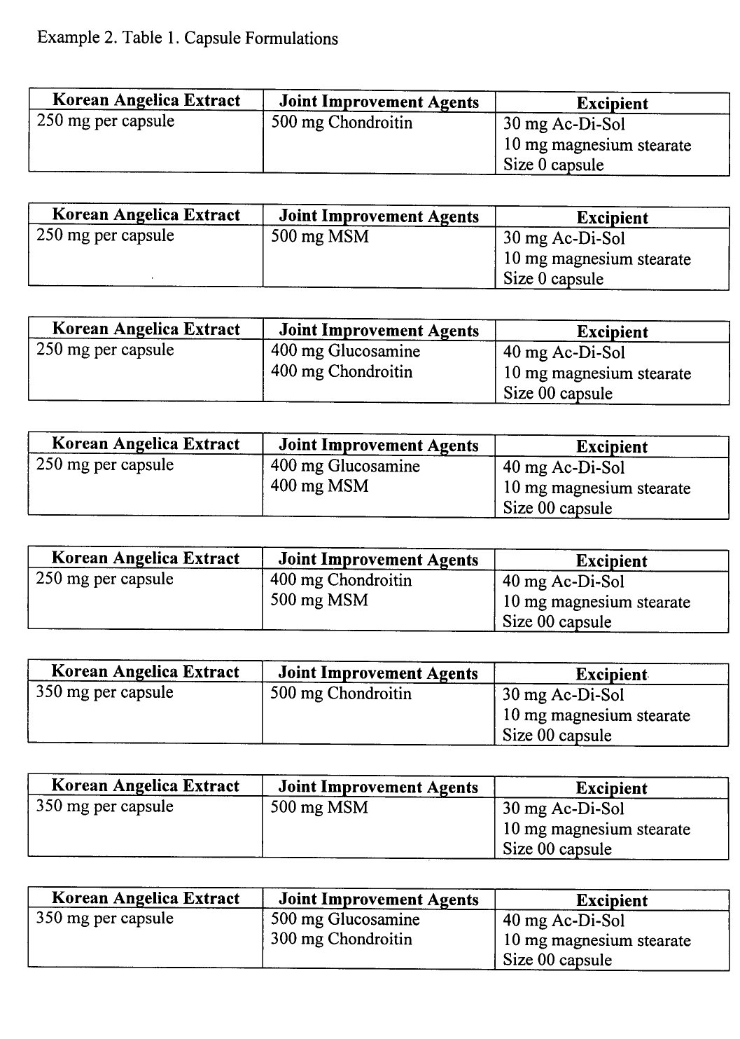

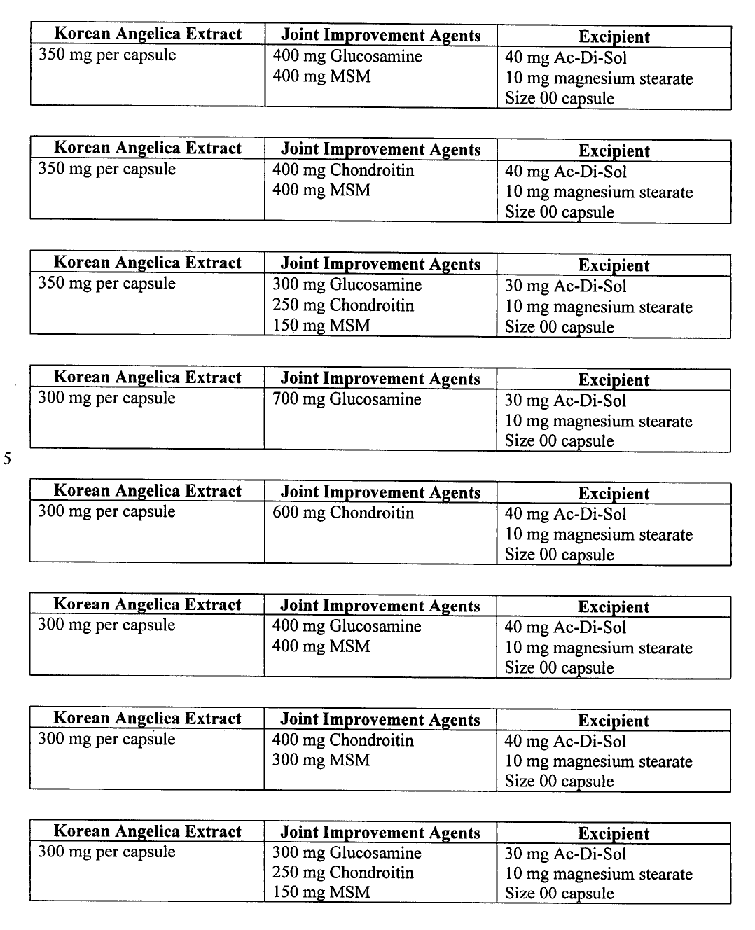

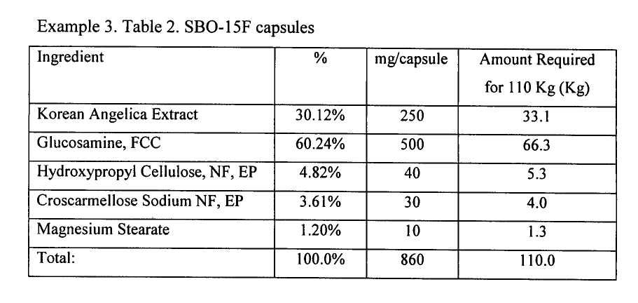

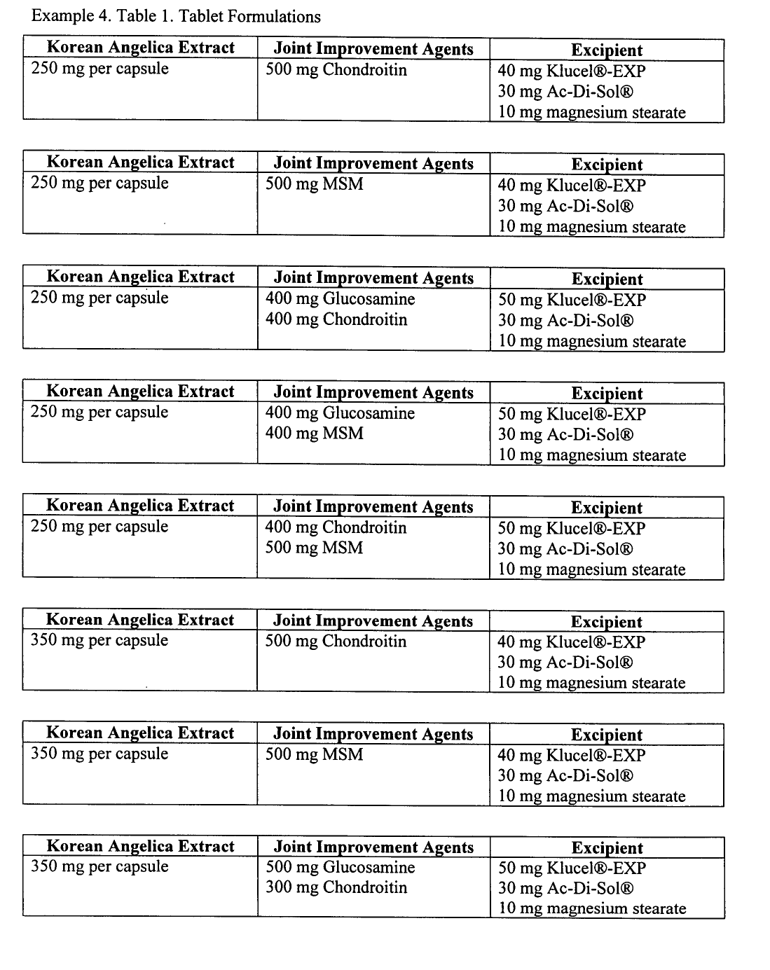

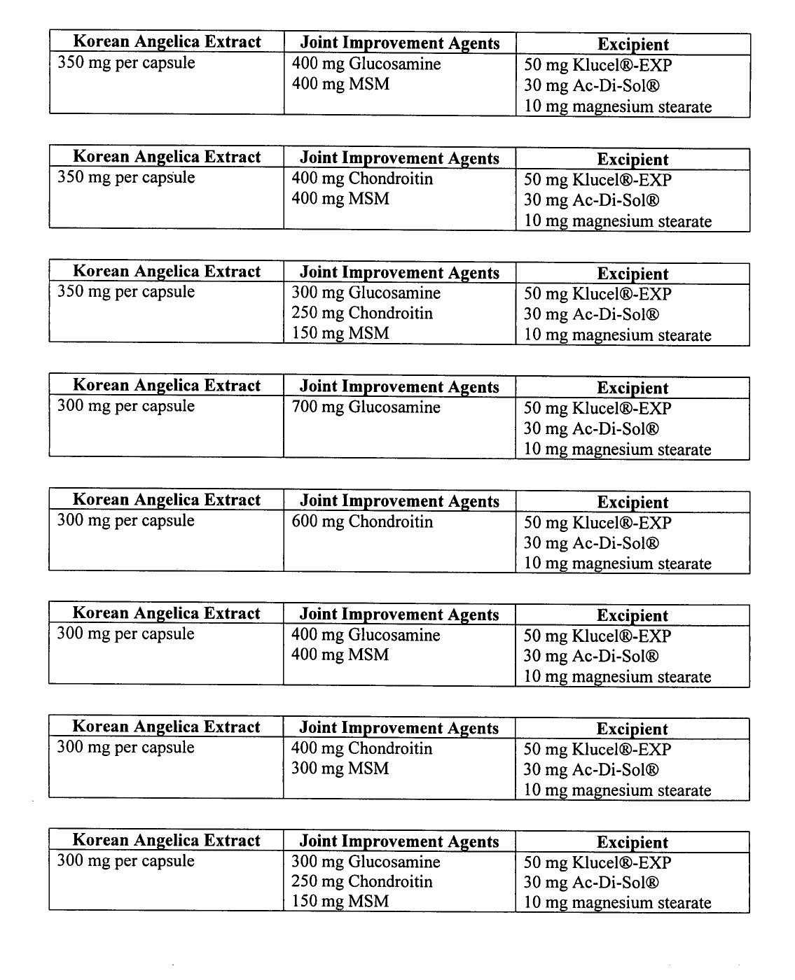

### BRIEF DESCRIPTION OF DRAWINGS NOTED IN THE BACK PAGES OF THE PATENT FILING AS FIGS. 1-5

### SUMMARY OF THE INVENTION

Provided herein are composition consisting of decursinol and its derivatives from extract of roots of plant Angelica gigas Nakai (Korean angelical and one or more joint improvement agents comprising Glucosamine sulfate, Chondroitin sulfate, and MSM.

The invention also provides a method of treating a subject with joint disorder and joint pain arising from it by administering a composition as provided above.

In various embodiments provided herein, the compositions are administered in a solid dosage form such as swallowable tablets, chewable tablets, hard gelatin capsule, soft-gel capsules, packets, sachet, and others.

In various embodiments of the present invention, the formulations may further comprise one or more excipients selected from erosion facilitators, diffusion facilitators, antioxidants, flavoring agents and carrier materials selected from binders, suspending agents, disintegration agents, filling agents, lubricants, diluents, anti-adherents, glidant, and antifoaming agents.

### DETAILED DESCRIPTION OF THE INVENTION

The interest in homeopathic and/or herbal medicines has increased recently due in part to the lower side effects associated with such medications. Homeopathy is commonly used to mean a system of medicine based on the use of infinitesimal doses of medicines capable of producing symptoms similar to those of the disease treated. By stimulating a subject's natural defenses (i.e., increasing the symptoms) the subject will be motivated or directed towards homeostasis, since one's symptoms are actually efforts of the organism to reestablish homeostasis or balance. Homeopathic treatment encompasses some forms of natural materials including plant extracts and the like. However, some natural plant extracts are not necessarily homeopathic treatments as the extracts themselves do not stimulate disease or disorder symptoms but rather inhibit their onset or severity.

It has been discovered that formulations of the extract of naturally occurring decursinol and its derivatives from a plant extract and one or more of the joint improvement agents exhibit synergetic effect in relieving the joint pain and reducing the inflammation without undesirable side effects. Methods of treatment using the formulations of the present invention are also described.

The invention provides methods and compositions that utilize oral dosage form of herbal medicines. The routes of administration used in the methods and compositions of the invention do not require excessive amounts of the herbal medicine or active ingredient, routes identified herein.

It is reported that decursinol and its related derivatives have anti-nociceptive properties and can alleviate the pain and inflammation in mammal. See, e.g., Seuong-Soo Choi et al., Antinociceptive Profiles of Crude Extract from Roots of Angelica gigas Nakai in Various Pain Models, Biol. Pharm. Bull, 26 (9): 1283-1288 (2003); S-S Choi et al., Antinociceptive mechanisms of orally administered decursinol in the mouse, Life Sciences, 73, 471-483 (2003).

### GLOSSARY

As used herein, the terms “comprising,” “including,” and “such as” are used in their open, non-limiting sense.

The term “about” is used synonymously with the term “approximately.” Illustratively, the use of the term “about” indicates that values slightly outside the cited values, i.e. dosages comprising plus or minus 0.1% to 10%, which are also effective and safe. As one of ordinary skill in the art would understand, such dosages are thus encompassed by the scope of the claims reciting the terms “about” and “approximately.”

“Anti-adherents,” “glidants,” or “anti-adhesion” agents prevent components of the formulation from aggregating or sticking and improve flow characteristics of a material. Such compounds include, e.g., colloidal silicon dioxide such as Cab-o-sil®; tribasic calcium phosphate, talc, corn starch, DL-leucine, sodium lauryl sulfate, magnesium stearate, calcium stearate, sodium stearate, kaolin, and micronized amorphous silicon dioxide (Syloid®) and the like.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, e.g., butylated hydroxytoluene (BHT), sodium ascorbate, and tocopherol.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

“Carrier materials” include any commonly used excipients in pharmaceutics and should be selected on the basis of compatibility with the and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “ly compatible carrier materials” may comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Sciences, Mack Publishing Co., Easton, Pa. 1975; Liberman, H. A. and Lachman, L., Eds., Dosage Forms, Marcel Decker, New York, N.Y., 1980; and Dosage Forms and Drug Delivery Systems, Seventh Ed. (Lippincott Williams & Wilkins 1999).

A “derivative” is a compound that is produced from another compound of similar structure by the replacement of substitution of an atom, molecule or group by another suitable atom, molecule or group. For example, one or more hydrogen atom of a compound may be substituted by one or more alkyl, acyl, amino, hydroxyl, halo, haloalkyl, aryl, heteroaryl, cycloaolkyl, heterocycloalkyl, or heteroalkyl group to produce a derivative of that compound.

“Diffusion facilitators” and “dispersing agents” include materials that control the diffusion of an aqueous fluid through a coating. Exemplary diffusion facilitators/dispersing agents include, e.g., hydrophilic polymers, electrolytes, Tween® 60 or 80, PEG and the like. Combinations of one or more erosion facilitator with one or more diffusion facilitator can also be used in the present invention.

“Diluents” increase bulk of the composition to facilitate compression. Such compounds include e.g., lactose; starch; mannitol; sorbitol; dextrose; microcrystalline cellulose such as Avicel®; dibasic calcium phosphate; dicalcium phosphate dihydrate; tricalcium phosphate; calcium phosphate; anhydrous lactose; spray-dried lactose; pregelatinzed starch; compressible sugar, such as Di-Pac® (Amstar); mannitol; hydroxypropylmethylcellulose; sucrose-based diluents; confectioner's sugar; monobasic calcium sulfate monohydrate; calcium sulfate dihydrate; calcium lactate trihydrate; dextrates; hydrolyzed cereal solids; amylose; powdered cellulose; calcium carbonate; glycine; kaolin; mannitol; sodium chloride; inositol; bentonite; and the like.

The term “disintegrate” includes both the dissolution and dispersion of the dosage form when contacted with gastrointestinal fluid.

“Disintegration agents” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” or “sweeteners” useful in the compositions of the present invention include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” are compounds that prevent, reduce or inhibit adhesion or friction of materials. Exemplary lubricants include, e.g., stearic acid; calcium hydroxide; talc; sodium stearyl fumerate; a hydrocarbon such as mineral oil, or hydrogenated vegetable oil such as hydrogenated soybean oil (Sterotex®); higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol or a methoxypolyethylene glycol such as Carbowax™, sodium oleate, glyceryl behenate, polyethylene glycol, magnesium or sodium lauryl sulfate, colloidal silica such as Syloid™, Carb-O-Sil®, a starch such as corn starch, silicone oil, a surfactant, and the like.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, and triacetin.

“Solubilizers” include compounds such as citric acid, succinic acid, fumaric acid, malic acid, tartaric acid, maleic acid, glutaric acid, sodium bicarbonate, sodium carbonate and the like.

“Stabilizers” include compounds such as any antioxidation agents, pH modifier, acidulants, and the like.

“Suspending agents” or “thickening agents” include compounds such as polyvinylpyrrolidone, e.g., polyvinylpyrrolidone K12, polyvinylpyrrolidone K17, polyvinylpyrrolidone K25, or polyvinylpyrrolidone K30; polyethylene glycol, e.g., the polyethylene glycol can have a molecular weight of about 300 to about 6000, or about 3350 to about 4000, or about 7000 to about 5400; sodium carboxymethylcellulose; methylcellulose; hydroxy-propylmethylcellulose; polysorbate-80; hydroxyethylcellulose; sodium alginate; gums, such as, e.g., gum tragacanth and gum acacia; guar gum; xanthans, including xanthan gum; sugars; cellulosics, such as, e.g., sodium carboxymethylcellulose, methylcellulose, sodium carboxymethylcellulose, hydroxypropylmethylcellulose, hydroxyethylcellulose; polysorbate-80; sodium alginate; polyethoxylated sorbitan monolaurate; polyethoxylated sorbitan monolaurate; povidone and the like.

“Surfactants” include compounds such as sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF); and the like.

A “therapeutically effective amount” or “effective amount” is that amount of a agent to achieve a pharmacological effect. The term “therapeutically effective amount” includes, for example, a prophylactically effective amount. An “effective amount” of a is an amount effective to achieve a desired pharmacologic effect or therapeutic improvement without undue adverse side effects. For example, an effective amount of a refers to an amount of that reduces acid secretion, or raises gastrointestinal fluid pH, or reduces gastrointestinal bleeding, or reduces the need for blood transfusion, or improves survival rate, or provides for a more rapid recovery from a acid related disorder. The effective amount of a agent will be selected by those skilled in the art depending on the particular patient and the disease level. It is understood that “an effect amount” or “a therapeutically effective amount” can vary from subject to subject, due to variation in metabolism of therapeutic agents such as s and/or prokinetic agents, age, weight, general condition of the subject, the condition being treated, the severity of the condition being treated, and the judgment of the prescribing physician.

“Treat” or “treatment” refers to any treatment of a disorder or disease associated with a gastrointestinal disorder, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent.”

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, and the like.

Joint Improvemt Agents

The composition of the invention comprises one or more joint improvement agents. A class of joint agent useful in the present invention include, e.g., Glucosamine, Chondroitin, and MSM (methylsulfonylmethane). In one embodiment, one or more joint improvement agent, when formulated with the plant medicament from Korean angelica, functions to substantially prevent or inhibit the erosion of the soft tissues surrounding the joint.

Treatment dosages generally may be titrated to optimize safety and efficacy. Typically, dosage-effect relationships from in vitro and/or in vivo tests initially can provide useful guidance on the proper doses for subject administration. Studies in animal models generally may be used for guidance regarding effective dosages for treatment of joint disorder in accordance with the present invention. In terms of treatment protocols, it should be appreciated that the dosage to be administered will depend on several factors, including the particular agent that is administered, the route chosen for administration, the condition of the particular subject.

In various embodiments, unit dosage forms for humans contain about 200 mg to about 800 mg, or preferably about 200 mg to 500 mg of dry powder of roots of Korean angelica plant.

In a further embodiment of the present invention, the formulation also contained one or more of joint improvement agents such as glucosamine, chondroitin, or methylsulfonylmethane (MSM) in amount that generally optimize safety and efficacy of its use. In various embodiments, unit dosage forms for humans contain about 150-1500 mg of these joint improvement agents.

In one embodiment, the unit dosage forms contain 200-800 mg of dry powder of Korean angelica plant and joint improvement agent glucosamine in about 150 mg, 300 mg, or about 500 mg, or about 800 mg.

In another embodiment, the unit dosage forms contain 200-800 mg of dry powder of Korean angelica plant and joint improvement agent chondroitin in about 150 mg, 300 mg, or about 500 mg, or about 800 mg.

Yet in another embodiment, the unit dosage forms contain about 150 mg, 300 mg, or about 500 mg, or about 800 mg of methylsulfonylmethane (MSM).

In another embodiment, the unit dosage forms contain 200-800 mg of dry powder of Korean angelica plant and mixture of more than one joint improvement agents in about 150-1500 mg.

Dosing Schedule

The elimination half life (T_{1/2}) of decursinol in mammal is about 3 hours. See Kim et al, Pharmacokinetic Study of Decursinol following Oral Administration in Rats, J. Kor. Pharm. Sci., 33 (3), 195-199 (2003). Considering this relatively short half-life of decursinol, the optimum dosing schedule of decursinol and its derivatives from the extract of Korean Angelica is twice daily, preferably in the morning and at night time. The dosage form is designed to deliver the therapeutic amount of decursinol and its derivatives from the extract of Korean Angelica and one or more of the joint improvement agents for a twice-a-day (or BID) dosing schedule.

Dosage Forms

The formulations of the present invention contain desired amounts of Korean angelica root extract and one or more of joint improvement agents and can be in the form of e.g. tablet; including a suspension tablet, a chewable tablet, or an effervescent tablet, or a pill; a powder such as a unit packaged powder in a sachet, a dispensable powder, and an effervescent powder; a capsule including both soft or hard gelatin capsules such as HPMC capsules. These formulations of the present invention can be manufactured by conventional pharmaceutical dosage form manufacturing techniques. Conventional dosage form manufacturing techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986).

Exemplary Solid Compositions

Solid compositions, e.g., tablets, chewable tablets, effervescent tablets, and capsules, are prepared by mixing the dry powder extract of Korean angelica with one or more joint improvement agents to form a bulk blend composition. When referring to these bulk blend compositions as homogeneous, it is meant that the plant extract powder is dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluent.

Compressed tablets are solid dosage forms prepared by compacting the bulk blend compositions described above. In various embodiments, compressed tablets of the present invention will comprise one or more flavoring agents. In other embodiments, the compressed tablets will comprise a film surrounding the final compressed tablet. In other embodiments, the compressed tablets comprise one or more excipients and/or flavoring agents.

A capsule may be prepared, e.g., by placing the bulk blend composition, described above, inside of a capsule. (including both soft and hard capsules, e.g., capsules made from animal-derived gelatin or plant-derived HPMC).

Exemplary Powder Compositions

A powder for suspension may be prepared by combining the dry powder extract of Korean angelica and one or more of the joint improvement agents. In various embodiments, the powder may comprise one or more excipients. In some embodiments of the present invention also comprise a flavoring agent, suspending agent and/or a wetting agent. A powder may be prepared by filling the bulk blend composition, described above, into an appropriate container system such as a unit dosage packet.

Many other types of solid dosage forms are available and known to those of ordinary skill in the art.

### EXAMPLES

The present invention is further illustrated by the following examples, which should not be construed as limiting in any way. The experimental procedures to generate the data shown are discussed in more detail below. For all formulations herein, multiple doses may be proportionally compounded as is known in the art.

The invention has been described in an illustrative manner, and it is to be understood that the terminology used is intended to be in the nature of description rather than of limitation.

### Example 1

### Capsule form of Decursinol and its Derivatives from Korean Angelica and Glucosamine Sulfate

The following specific formulations are provided by way of illustrating the present invention and are not intended to be limiting. The capsules were prepared by blending the dry powder of decursinol and its derivatives from the extract of Korean Angelica roots with the indicated amount of components shown in Table 1. Once the extract powder containing decursinol and its derivatives were homogeneously blended with the excipients, the appropriate weight of composition was filled into hard gelatin capsules, size 0 or 00, using an automatic encapsulators such as H&K 1200.

In particular, a 110 kg blend for manufacturing SBO-10E capsules according to the present invention was manufactured by the following procedure: First, about half of the extract powder was blended with glucosamine in a 5 cubic foot V-blender to form a pre-blend. Next, the remaining half of the extract powder and the croscarmellose sodium was added to the pre-blend, and the resulting mixture was blended until homogeneous powder was achieved. Lastly, magnesium stearate was added to the mixture and the mixture was mixed for 3 minutes before being discharged into a drum. The mixture was then encapsulated in size 0 hard gelatin capsules on an automatic encapsulators (H&K 1200). The amounts of the extract powder and excipients used in this example are set forth in table 7E, SBO-10E Capsules (see configurations/table in back pages of filing).

### Example 2

### Capsule Formulations

The following formulations are provided by way of reference only and are not intended to limit the scope of the invention. Each formulation contains therapeutically effective amount of Korean Angelica Extract and one or more of joint improvement agents. These formulations are manufactured by using the process shown in Example 2. Table 1. Capsule Formulations (see configurations/table in back pages of patent filing).

### Example 3

### Tablet Formulations of Decursinol and its Derivatives from Korean Angelica and Glucosamine Sulfate

The following specific formulations are provided by way of illustrating the present invention and are not intended to be limiting. The tablets were prepared by blending the dry powder of decursinol and its derivatives from the extract of Korean Angelica roots with the indicated amount of components shown in Table 1. Once the extract powder containing decursinol and its derivatives were homogeneously blended with the excipients, the appropriate weight of composition was compressed using shaped tooling (oval) to form capsule shaped tablets or caplets. Variety of tablet press such as Fette, Korsch, Manasty or Beta press may be used.

In particular, a 110 kg blend for manufacturing SBO-15E tablets according to the present invention was manufactured by the following procedure: First, about half of the extract powder was blended with glucosamine in a 5 cubic foot V-blender to form a pre-blend. Next, the remaining half of the extract powder, HPC (Klucel®-EXF, Aqualon), croscarmellose sodium (Ac-di-Sol®, FMC) were added to the pre-blend, and the resulting mixture was blended until homogeneous powder was achieved. Lastly, magnesium stearate was added to the mixture and the mixture was mixed for 3 minutes before being discharged into a drum. The mixture was then compressed to the right unit weight caplets using concave beveled-edged oval shaped tooling (11 mm diameter×17 mm length) mounted in Fette tablet press. The hardness of the tablet is 10-12 kP and the friability is less than 1%. The amounts of the extract powder and excipients used in this example are set forth in Example 3. Table 2. SBO-15F capsules (see configurations/table in back pages of filing)

### Example 4

### Tablet Formulations

The following formulations are provided by way of reference only and are not intended to limit the scope of the invention. Each formulation contains therapeutically effective amount of Korean Angelica Extract and one or more of joint improvement agents. These formulations are manufactured by using the process shown in Example 4. Table 1. Tablet Formulations (see configurations/table in back pages of filing)

What is claimed is:

1. A formulation having synergetic joint pain relief properties, comprising:
(a) dry powder of extract from roots of Korean Angelica (Angelica gigas Nakai); and
(b) at least one joint improvement agents

2. A formulation according to claim 1, wherein the active ingredients are decursinol and its derivatives including decursin.

3. A formulation according to claim 1, wherein the joint improvement agent is selected from glucosamine, chondroitin, methylsulfonylmethane (MSM), or their salts.

4. A formulation according to claim 1 comprising about 200 mgs to about 800 mgs of dry powder of extract from roots of Korean Angelica (Angelica gigas Nakai).

5. A formulation according to claim 1 comprising about 200 mgs, or about 300 mgs, or about 300 mgs, or about 400 mgs, or about 500 mgs of dry powder of extract from roots of Korean Angelica (Angelica gigas Nakai).

6. A formulation according to claim 1, wherein the joint improvement agent is glucosamine or its salt form.

7. A formulation according to claim 1, wherein the joint improvement agent is chondroitin or its salt form.

8. A formulation according to claim 1, wherein the joint improvement agent is dimethylsulfoxide (MSM) or its salt form.

9. A formulation according to claim 1, wherein the joint improvement agent is glucosamine and chondroitin or their respective salt forms.

10. A formulation according to claim 1, wherein the joint improvement agent is glucosamine and methylsulfonylmethane (MSM) or their respective salt forms.

11. A formulation according to claim 1, wherein the joint improvement agent is glucosamine, chondroitin and methylsulfonylmethane (MSM) or their respective salt forms.

12. A formulation according to claim 1, wherein the amount of joint improvement agents is 150-800 mg.
